(19) United States
(12) Patent Application Publication
(10) Pub. No.: US 2018/0043122 A1
(43) Pub. Date: Feb. 15, 2018

(54) Customized CPAP Masks and Related Modeling Algorithms

(71) Applicant: MetaMason, Inc., Los Angeles, CA (US)

(72) Inventors: Ryan Michael Oenning, Los Angeles, CA (US); Charles Lucien Pyott, Los Angeles, CA (US)

(73) Assignee: MetaMason, Inc., Los Angeles, CA (US)

(21) Appl. No.: 15/675,597

(22) Filed: Aug. 11, 2017

Related U.S. Application Data
(60) Provisional application No. 62/373,866, filed on Aug. 11, 2016.

(51) Int. Cl. A61M 16/06; A61M 16/08; A62B 18/02

(52) U.S. Cl. CPC A61M 16/0605; A62B 18/02; A61M 16/08; A61M 2016/0661; A61M 2207/00; B29L 2031/4835

(57) ABSTRACT

Customized CPAP masks and related modeling algorithms in accordance with embodiments of the invention are illustrated. One embodiment includes a method for generating a surface model of a CPAP mask, the method including obtaining facial scan data of a user's face, determining a set of landmark points from the facial scan data, wherein the set of landmark points relate to anatomical locations on the user's face, generating a base model, and deforming the base model into a deformed model using the set of landmark points.

### CROSS-REFERENCE TO RELATED APPLICATIONS

The current application claims the benefit of and priority under 35 U.S.C. §119(e) to U.S. Provisional Patent Application No. 62/373,866 entitled “CPAP Mask Algorithms” filed Aug. 11, 2016. The disclosure of U.S. Provisional Patent Application No. 62/373,866 is hereby incorporated by reference in its entirety for all purposes.

### FIELD OF THE INVENTION

The present invention generally relates to customized apparel and, more specifically, customized CPAP Masks and related modeling algorithms.

### BACKGROUND

Sleep apnea is a sleep disorder characterized by pauses in breathing or periods of shallow breathing during sleep. People affected by sleep apnea can experience sleepiness or feel tired during the day due to the disruptions to their normal sleep. Continuous positive airway pressure (“CPAP”) is a treatment option for obstructive sleep apnea. CPAP can be implemented as a positive airway pressure ventilator, which can apply air pressure on a continuous basis to keep the airways continuously open. Common implementations of CPAP therapy involve a nasal mask.

### SUMMARY OF THE INVENTION

Customized CPAP masks and related modeling algorithms in accordance with embodiments of the invention are illustrated. One embodiment includes a method for generating a surface model of a CPAP mask, the method including obtaining facial scan data of a user's face, determining a set of landmark points from the facial scan data, wherein the set of landmark points relate to anatomical locations on the user's face, generating a base model, and deforming the base model into a deformed model using the set of landmark points.

In another embodiment, the method further includes generating a customized model by altering the deformed model using the facial scan data.

In a further embodiment, altering the deformed model includes performing a Boolean operation using the deformed model and a surface model generated using the facial scan data to alter an inner boundary surface of the deformed model such that the inner boundary surface of the deformed model can sit flush against the user's face.

In still another embodiment, the method further includes manufacturing the customized model using an additive manufacturing process.

In a still further embodiment, the method further includes generating a mold model based on the customized model.

In yet another embodiment, the base model is a rigged model including a plurality of joints that deforms the base model when moved.

In a yet further embodiment, deforming the base model includes moving at least one of the plurality of joints based on a predefined relationship with at least one of the set of landmark points.

In another additional embodiment, the base model is deformed in adherence to a set of constraints.

In a further additional embodiment, the base model includes an averaged model obtained from an aggregate database, wherein the averaged model is an average of stored facial scan data in the aggregate database.

In another embodiment again, the averaged model is an average of stored facial scan data of a selected demographic.

In a further embodiment again, further including determining a nasal curve from the facial scan data and altering nasal fittings of the deformed model to match the nasal curve.

In still yet another embodiment, determining a nasal curve includes manually determining the nasal curve.

In a still yet further embodiment, the deformed model includes a nasal interface including a single slit.

In still another additional embodiment, the deformed model includes a nasal interface including asymmetric nasal fittings.

In a still further additional embodiment, the asymmetric nasal fittings each includes a flange.

In still another embodiment again, obtaining facial scan data includes scanning the user's face using scanning technology.

In a still further embodiment again, obtaining facial scan data includes retrieving the facial scan data from a remote server.

In yet another additional embodiment, determining a set of landmark points includes manually determining the set of landmark points.

In a yet further additional embodiment, determining a set of landmark points includes using an algorithm and machine vision to automatically determine the set of landmark points.

Yet another embodiment again includes a customized CPAP mask including a customized mask comprising asymmetric nasal fittings and an inner boundary surface customized to sit flush with a face of a user, a tube frame connected to the customized mask, wherein the tube frame comprises slots for strap attachment, and a tube gasket connected to the tube frame, wherein the tube gasket is asymmetrical.

In a yet further embodiment again, the asymmetric nasal fittings includes a flange, wherein the flange is designed to expand when positive pressure is applied to the inside of the nasal fittings.

In another additional embodiment again, the customized mask further includes a gasket side, wherein the gasket side houses the connection point for the tube frame, and wherein the center perpendicular line of the gasket side crosses a specific distance above a top ear point of the user when the customized mask is worn by the user.

In a further additional embodiment again, the customized mask further includes a bottom surface, wherein the bottom surface meets the face of the user at a point between the user's upper lip and columella when the customized mask is worn by the user.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 conceptually illustrates a process for generating a customized model in accordance with an embodiment of the invention.

FIG. 2 conceptually illustrates a surface model of a face scan in accordance with an embodiment of the invention.

FIG. 3 conceptually illustrates landmark points used for mask deformation in accordance with an embodiment of the invention.

FIG. 4 conceptually illustrates aggregation of scan data to produce base models in accordance with an embodiment of the invention.

FIG. 5 conceptually illustrates an analysis for determining new product categories in accordance with an embodiment of the invention.

FIG. 6 conceptually illustrates using a base rigged model and landmark points data to generate a deformed mask in accordance with an embodiment of the invention.

FIG. 7 conceptually illustrates a process for deforming a base rigged model in accordance with an embodiment of the invention.

FIG. 8 conceptually illustrates deformation constraints in accordance with an embodiment of the invention.

FIG. 9 conceptually illustrates deformation constraints for determining initial mask placement and strap angle in accordance with an embodiment of the invention.

FIGS. 10A-10B conceptually illustrate the generation of an inner boundary control surface in accordance with an embodiment of the invention.

FIGS. 11A-11B conceptually illustrate the generation of nasal curves in accordance with an embodiment of the invention.

FIGS. 12A-12D conceptually illustrates various nasal fitting designs in accordance with several embodiments of the invention.

FIG. 13A conceptually illustrates profile views of various nasal fitting designs in accordance with several embodiments of the invention.

FIG. 13B conceptually illustrates effects of positive pressure on a nasal fitting in accordance with an embodiment of the invention.

FIG. 14A conceptually illustrates a dual pronged nasal interface of a CPAP mask in accordance with an embodiment of the invention.

FIG. 14B conceptually illustrates a single slit nasal interface of a CPAP mask in accordance with an embodiment of the invention.

FIGS. 15A and 15B conceptually illustrate exploded views of different CPAP mask configurations in accordance with various embodiment of the invention.

### DETAILED DESCRIPTION

Turning now to the drawings, customized CPAP masks and related modeling algorithms are illustrated. In many embodiments of the present invention, a CPAP mask customized to fit a specific face can be fabricated using a computer generated three-dimensional (“3D”) model and various manufacturing techniques. The 3D model can be generated using modeling algorithms that takes in facial scan data to create a mask model customized to a specific face of a user. In some embodiments, the modeling algorithm generates landmark points using facial scan data. These landmark points can be used in conjunction with a deformation bone system to alter a base shell model into a deformed model, which can then be used to form a customized mask model.

In some embodiments, a 3D model of a customized CPAP mask can be used in conjunction with manufacturing methods, such as but not limited to additive manufacturing processes, to fabricate a customized CPAP mask. In other embodiments, a 3D model of a customized CPAP mask is used to generate a 3D model of a CPAP mask mold, which can be fabricated using a variety of manufacturing methods, such as but not limited to additive manufacturing processes. A customized CPAP mask can be manufactured using the fabricated CPAP mask mold through a variety of manufacturing methods, such as but not limited to injection molding. Modeling algorithms to create 3D customized models of CPAP masks and different CPAP mask configurations are further described below.

### CPAP Mask Modeling Algorithms

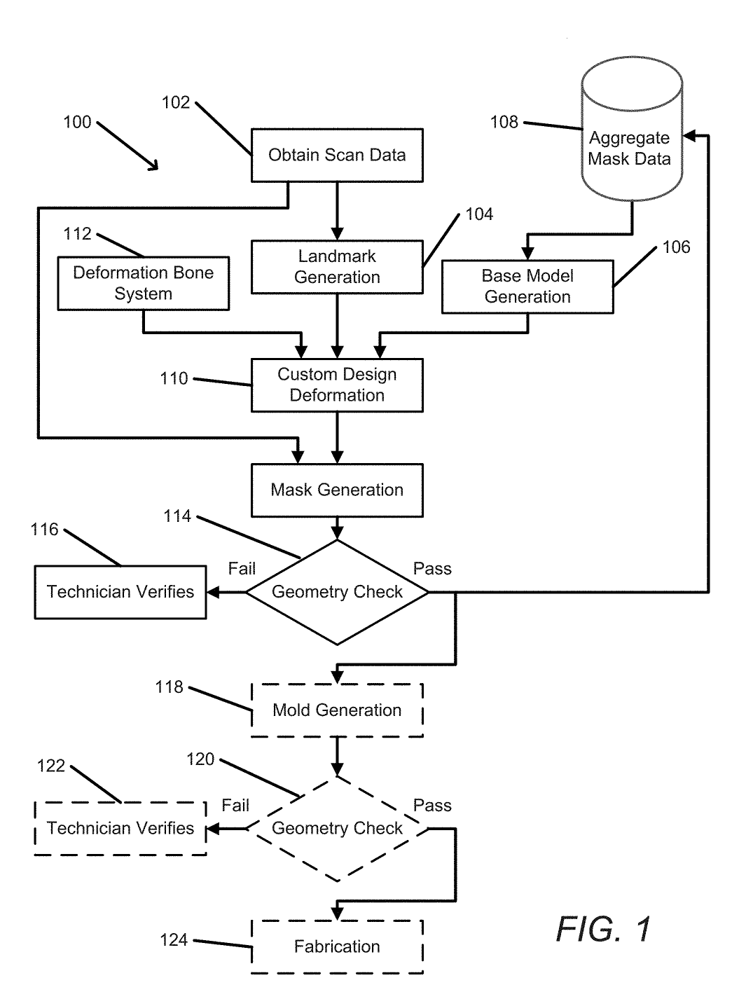

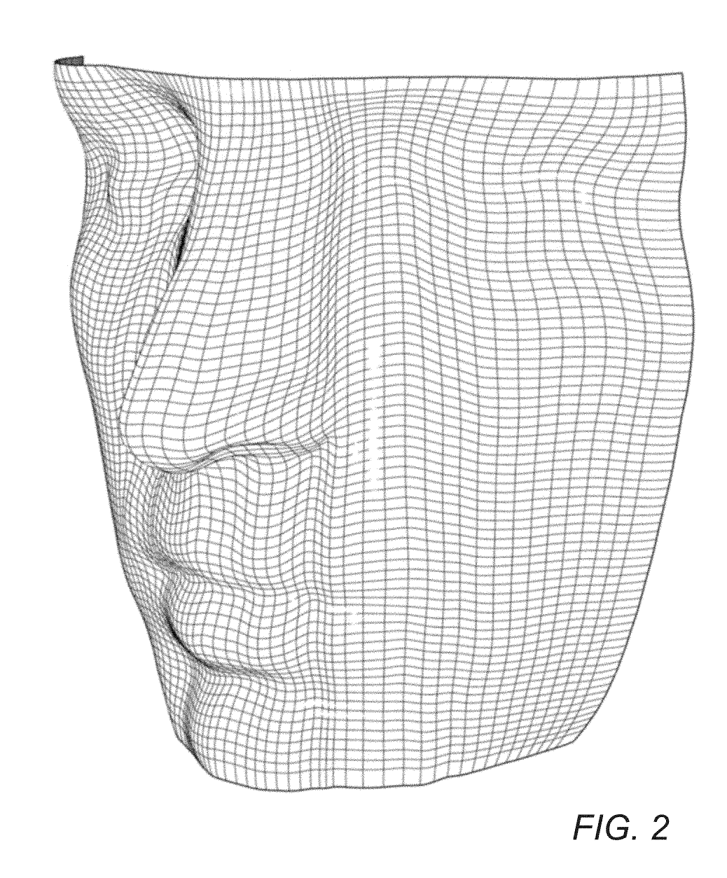

A process 100 that can be utilized for generating a customized mask model in accordance with an embodiment of the invention is illustrated in FIG. 1. In the exemplary embodiment, the process 100 starts with obtaining facial scan data (102). Facial scan data can be obtained using any of a number of ways. In many embodiments, facial scan data can be obtained as a data file, such as but not limited to CAD files and 3D modeling files. The facial scan data can be obtained or retrieved locally, from a remote server, or any of a number of sources. In some embodiments, facial scan data is obtained in real-time by scanning a face using 3D scanning technology. FIG. 2 conceptually illustrates a surface model that can be generated from facial scan data. Although FIG. 2 shows a specific type of modeling to represent curves and surfaces, a person having ordinary skill in the art would appreciate that any of a number of modeling systems can be used, such as but not limited to polygon meshes, parametrics, non-uniform rational B-splines (“NURBS”), and voxels. Furthermore, it could readily be appreciated that the variety of modeling systems can be applied and percolated to all of the remaining steps of the modeling algorithms. Accordingly, the modeling algorithms can be adjusted to reflect the model used.

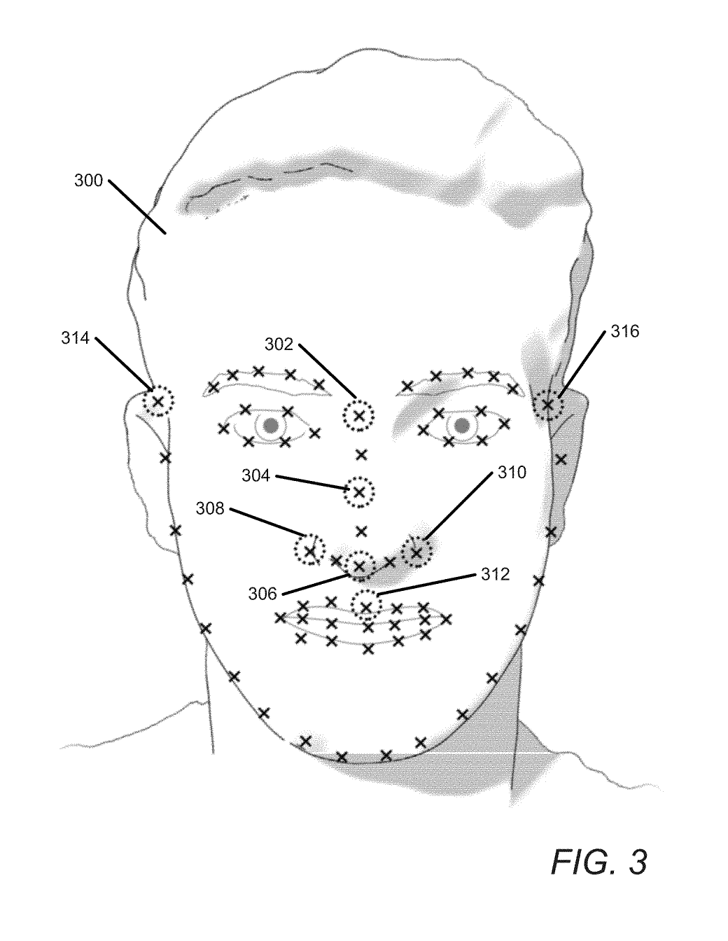

Once facial scan data is obtained, landmark points can be determined (104). Landmark points can be defined and determined in a number of different ways. In many embodiments, the landmark points are a set of points in 3D space that correlates with a surface model generated by the facial scan data. In some embodiments, landmark points are predefined to associate with anatomical locations on a face. FIG. 3 conceptually illustrates landmark points on a human face 300. In the illustrative embodiment, the set of landmark points includes the bridge of the nose 302, the middle of the nose 304, the nose tip 306, right ala 308, left ala 310, upper lip 312, right ear top 314, and left ear top 316. The set of landmark points can be determined through a number of different ways. In various embodiments, the landmark points are manually determined. For example, a technician can manually select points in 3D space on a surface model generated by facial scan data. In other embodiments, the set of landmark points are determined automatically by the computer through facial recognition algorithms. In several embodiments, a hybrid manual/automatic scheme can be used to determine the set of landmark points. A computer program can suggest the landmark points, which can then be confirmed or altered by a technician.

The process 100 can further include generating (106) a base model. In many embodiments, the base model is a surface model that is to be deformed. The base model can be generated in a number of different ways. In some embodiments, the base model can be dynamically generated from a database (108) that aggregates historical facial scan data. In further embodiments, the base model can be generated based on several variables, such as but not limited to demographic information of the person for which the CPAP mask is customized. In other embodiments, the base model is a constant, generic model generated from a data file that can be used for all situations. Methods of generating a base model are discussed in further detail below.

The base model can be deformed (110) into a deformed model. In many embodiments, the base model is deformed using determined landmark points. In further embodiments, the base model is a rigged model and is deformed using a deformation bone system (112). A rigged model can be defined as a 3D model bound to a digital skeleton that includes bones and joints. When the bones and joints are moved, the surface of the 3D model is deformed accordingly. In some embodiments, the base model is deformed to match a surface model generated from facial scan data. Methods of deforming a base model are discussed in further detail below. Once the deformed model is generated, a mask model customized to a face of a specific person can be generated. The customized mask model can be generated using the deformed model and facial scan data. In several embodiments, the deformed model is altered to match a surface model generated using facial scan data, ensuring that the customized mask model is tailored to a user's face. After generation of a customized mask model, the geometry of the model can optionally be verified (114) to ensure proper quality control. In many embodiments, the geometry of the model is automatically checked by a program to determine if the geometry meets certain constraint criteria. If the geometry check fails, a technician can verify (116) whether the failure is harmless. In other embodiments, the geometry is manually checked by a technician.

The process 100 can optionally include a manufacturing step. A customized mask model can be fabricated using a variety of different methods. In many embodiments, the customized mask model is converted into a 3D printer readable file, such as but not limited to STL files. The customized mask model can then be fabricated using an additive manufacturing process. In the exemplary embodiment shown in FIG. 1, the customized mask model can optionally be fabricated using a molding process. Using the customized mask model, a mold model can be generated (118). A geometry check (120) can optionally be performed, similar to the geometry check above, which can include verification (122) from a technician. Once the mold model is satisfactory, the mold model can be fabricated (124) using any of a variety of manufacturing methods, such as but not limited to additive manufacturing processes. Molds generated using additive manufacturing processes allows for quick and inexpensive fabrication. This facilitates the use of investment molding techniques.

Although a specific modeling algorithm is discussed above with respect to FIG. 1, a person having ordinary skill in the art would readily appreciate that the algorithm discussed can be altered as appropriate to the requirements of a given application. For example, a customized mask model can be fabricated directly without the need of generating a mold model and fabricating a mold. Below are discussions in further detail of the steps described above.

### Base Mask Model Generation

In many embodiments, the bone deformation system is an automatic process where the quality of the end result depends on the quality of the base mask model. A base mask model can be generated using a number of different methods. In several embodiments, a static, generic base model is used for all cases. In many embodiments, an aggregate database is kept of all past facial scan data. The facial scan data can contain attributes that describes information concerning the user to which the scan data belongs. For example, in some embodiments, the aggregate database contains facial scan data that are classified by a number of different demographic information, such as but not limited to age, gender, and ethnicity. Using such a database, a base mask model can be generated to target a user's demographic. FIG. 4 conceptually illustrates a graphical interface depicting the process of generating a base mask model targeting a user's demographic. Once the attributes are selected, the aggregate database can produce a base mask model that is the average of all the facial scan data fitting the selected criteria.

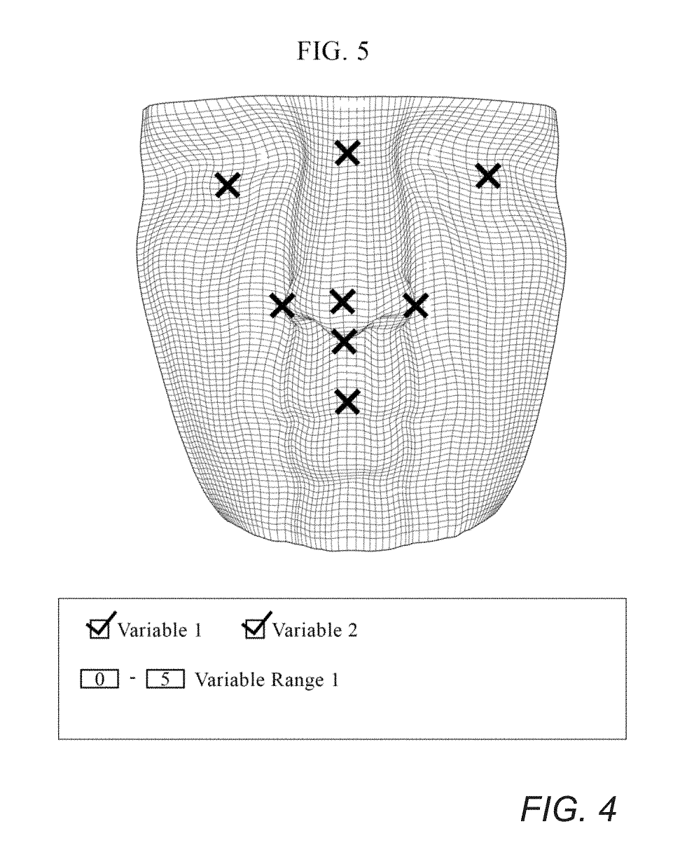

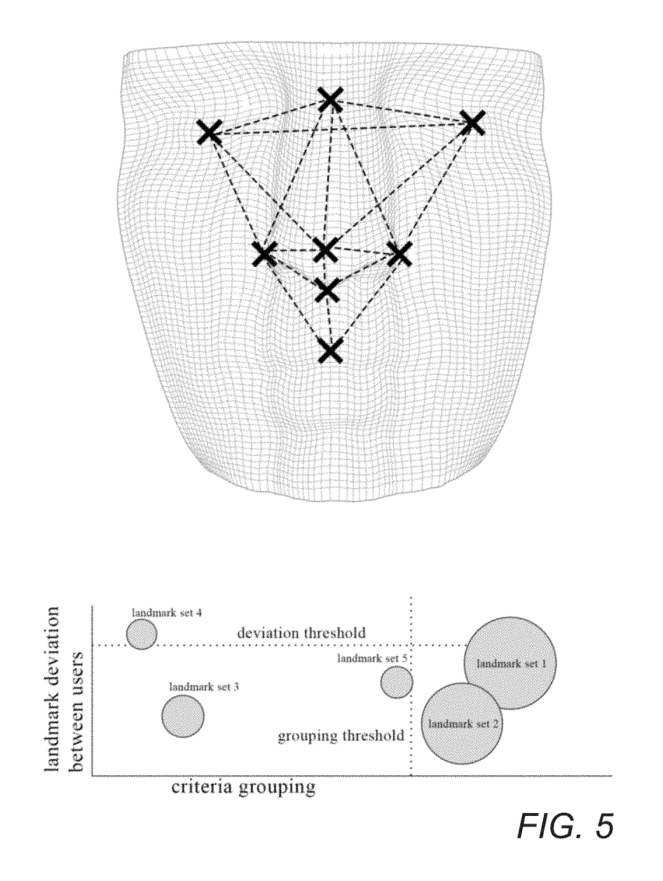

In many embodiments, the aggregate database stores a base mask model that is the average of previous facial scan data. The base mask model can be updated as new facial scan data is entered. In some embodiments, a number of different mask models is stored in the aggregate database. The different base mask models can represent different categories of masks based on any of a number of factors, such as but not limited to age, gender, and ethnicity. In several embodiments, the different base mask models can represent different categories of face shape. The database can aggregate information and determine different criteria groupings and deviations of landmark points among users. Analyzing the deviation among landmarks across different user groups can provide information on when to create a new base mask model. FIG. 5 conceptually illustrates how a database can determine when a new base mask model should be created. As shown, when the different subsets of landmark points deviate above a threshold, a new base mask model category can be created.

Although the discussions above relate to specific implementations of generating base mask models, a person having ordinary skill in the art would appreciate that the implementations described above can be altered to fit certain requirements of a given application. For example, the aggregate database can aggregate facial scan data to store surface models of a user's face instead of base mask models as described above.

Mask Model Deformation

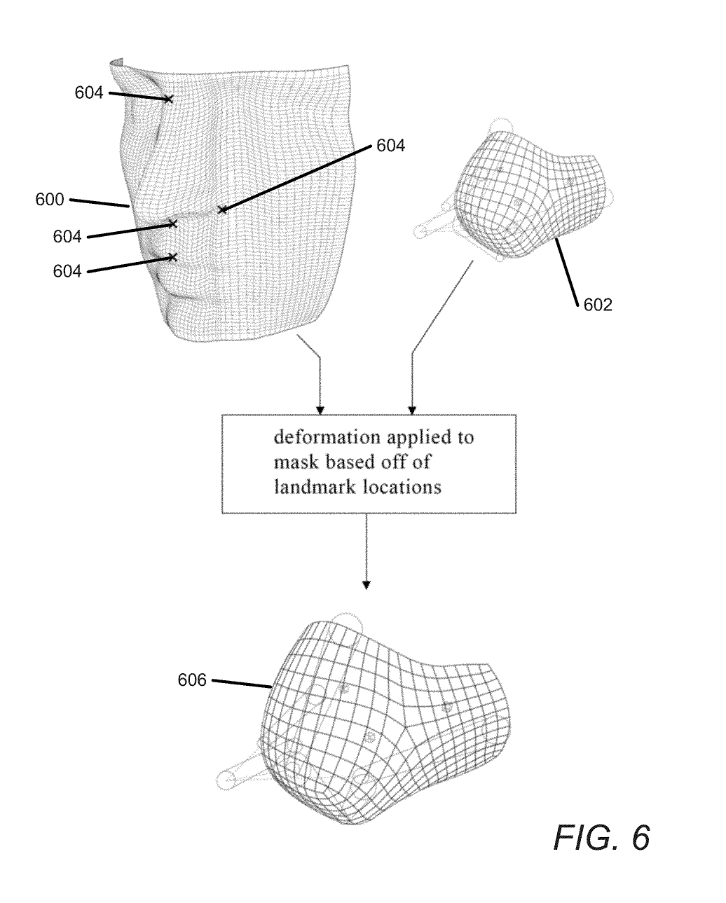

A base mask model can be deformed based on landmark points determined from facial scan data. FIG. 6 conceptually illustrates deforming 600 a base mask model 602 using landmark points 604 determined from facial scan data to generate a deformed mask model 606. Many methods can be used to deform a base mask model. In some embodiments, the base mask model is deformed to fit curves generated by facial scan data and the associated landmark points. In other embodiments, the base mask model is a rigged model that can be deformed based on landmark points. As described previously, rigged models contains bones and joints that can be moved, which deforms the model, including the surface of the model. Landmark points and bone joints can be associated to have relationships that define the deformations. The associations between bone joints and landmark points can be defined in a number of different ways. For example, a landmark point can be associated to a bone joint such that the two objects maintain a certain fit distance. Other and more complex relationships, including but not limited to mathematical relationships and equations, can also be defined. Different landmark points and different bone joints can be associated to have different relationships, which can depend on the specific landmark point and/or bone joint. Furthermore, specific areas of the mask model can be defined to have different weights in terms of the influence that each bone joint has on the respective area. FIG. 7 conceptually illustrates how landmark points can be used to deform a rigged base mask model. In the illustrated embodiment, bone joint 700 is associated with landmark point 702. As shown in FIG. 7, when landmark point 702 is moved either up 704 or down 706, bone joint 700 is moved accordingly to maintain a certain fit distance.

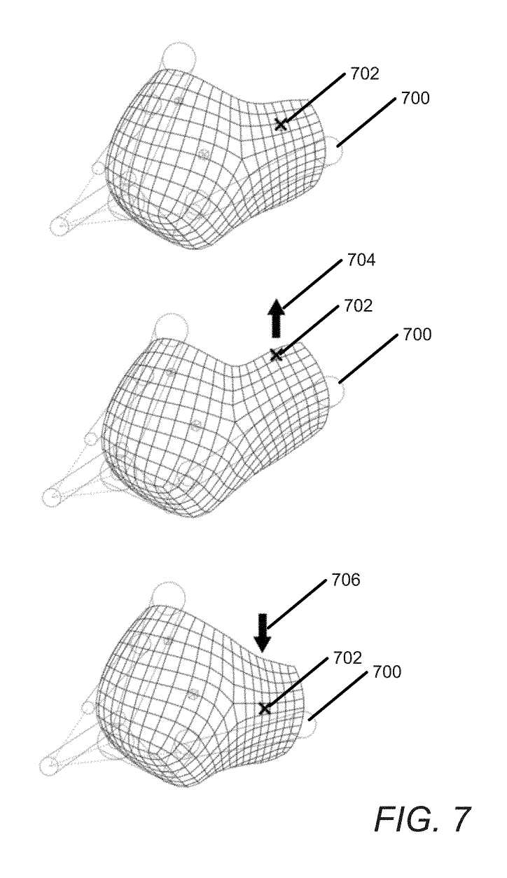

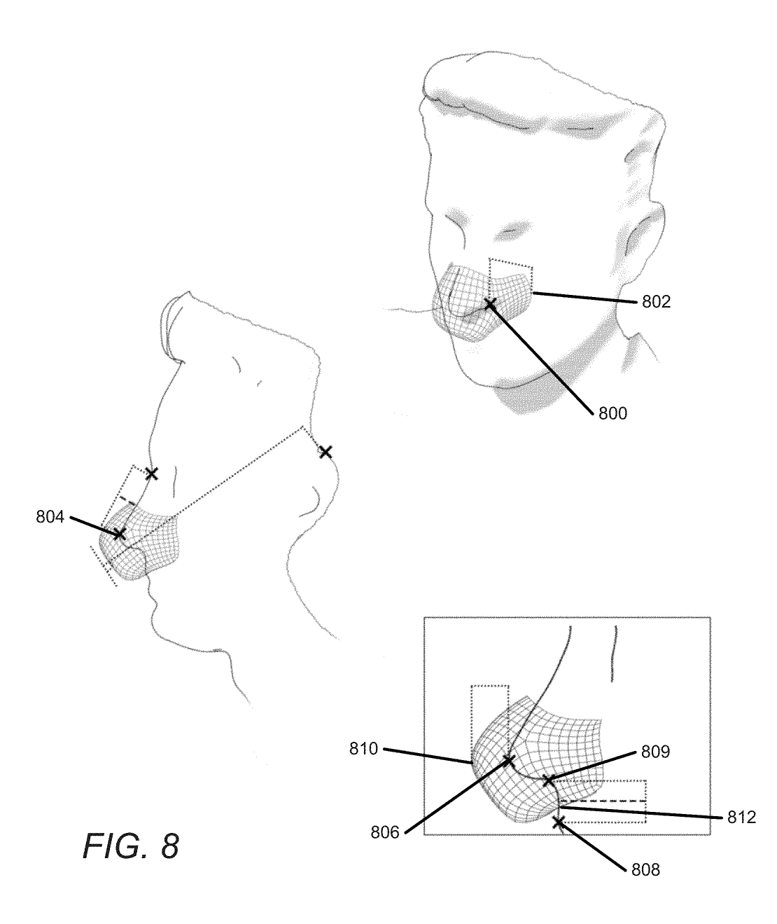

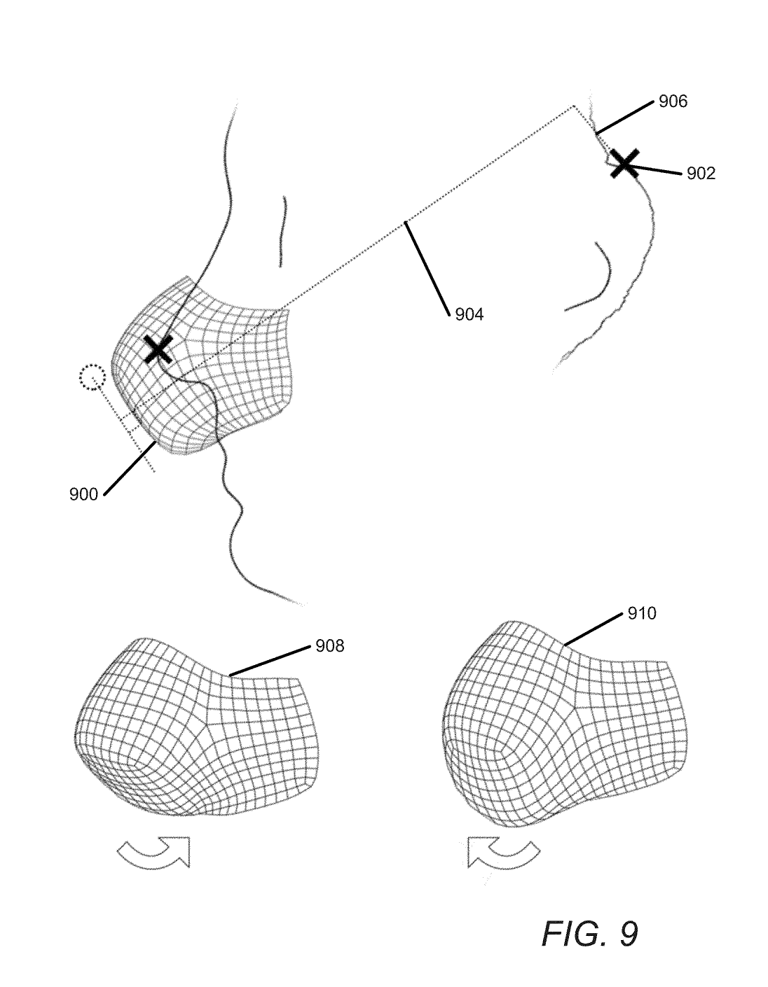

FIG. 8 conceptually illustrates several relationships/constraints between the base mask model and landmark points in accordance with an embodiment of the invention. As shown, landmark point 800, the left ala, determines the end 802 of the respective wing of the mask model. Landmark point 804, the nose tip, determines the position of the pivot point, which is defined to be a specific distance away in front of the landmark point 804. Landmark point 806, the nose tip, determines the offset of the pivot point 810. Landmark points 808, the upper lip, and 809, columella, defines how far the bottom surface 812 reaches on the user's face. In the illustrated embodiment, the bottom surface is defined to be between landmark points 808 and 809. FIG. 9 conceptually illustrates the relationship between the angle of the gasket side 900 and landmark point 902, the left ear top. The gasket side of a CPAP mask model can refer to the side where a tube gasket can be attached. In the illustrative embodiment, the angle is defined such that the perpendicular center line 904 of the gasket side 900 crosses a specific distance 906 above landmark point 902. Depending on the landmark point 902, the angle at which the gasket side 900 sits with respect to the user's face can vary, as shown in mask models 908 and 910. Although FIGS. 8 and 9 describe specific relationships/constraints among landmark points and the mask model, it can readily be appreciated that any kind of relationships/constraints can be defined as appropriate to the requirements of a given application.

Although mask deformation is described above with respect to methods of deforming mask models, a person having ordinary skill in the art would appreciate that the discussions above also applies to customized masks with features crafted from deformation constraints. As such, various embodiments of the invention are directed towards masks having features related to deformation constraints. For example, in some embodiments, a customized CPAP mask can have a gasket side angle such that the perpendicular center line crosses a specific distance above a specific ear of a user.

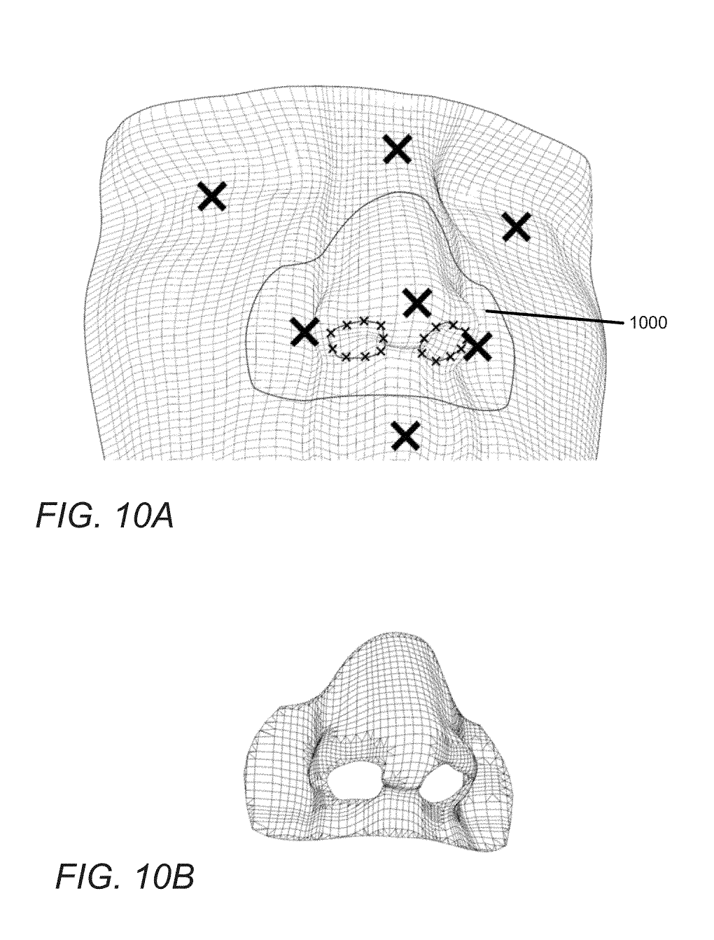

The mask model can also be altered using facial scan data. In many embodiments, facial scan data are used to produce a surface model of a user's face in order to customize the mask model for the user's face. In some embodiments, a Boolean operation is performed on a mask model and a surface model of a user's face to alter the mask model such that the mask model will sit flush against the user's face. FIG. 10A conceptually illustrates a surface model generated by facial scan data. As shown, area 1000 shows the section which would intersect with a mask model. FIG. 10B shows the excised section.

### Nasal Fittings

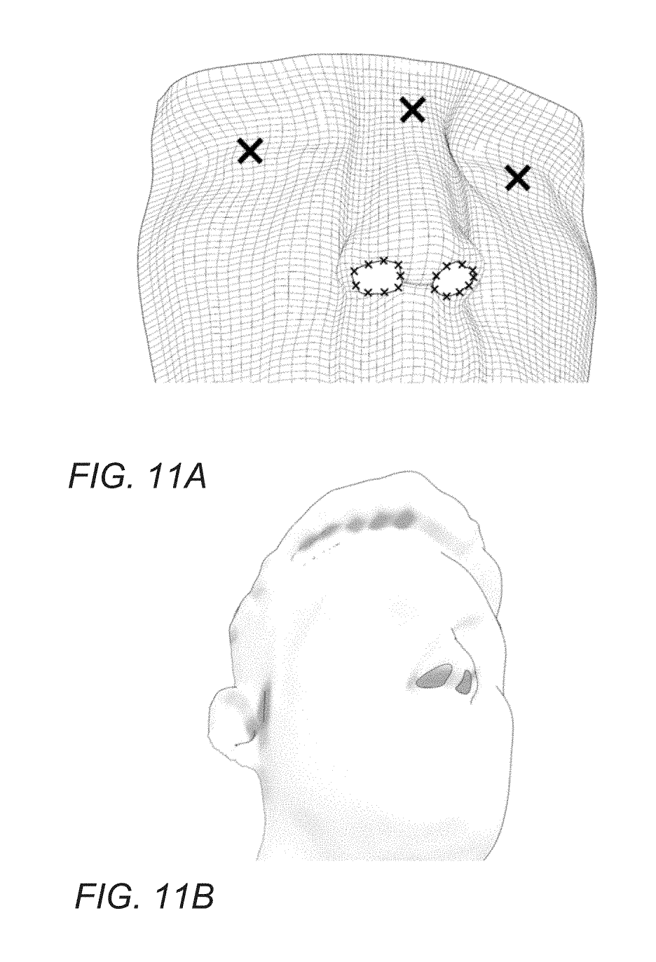

One aspect of CPAP masks is how well the nasal fittings fit in a user's nostrils. Customized CPAP masks in accordance with many embodiments of the invention can be designed to incorporate nasal fittings that are customized to fit a user's nostrils. There are many methods to design customized nasal fittings. In some embodiments, the mask models are automatically altered to incorporate customized nasal fittings using photographic images, facial scan data, and/or surface models. A curve tracing algorithm can determine nostril curves using the aforementioned assets and alter the mask model accordingly. In other embodiments, incorporating customized nasal fittings is manually shaped using photographic images, facial scan data, and/or surface models. FIGS. 11A and 11B conceptually illustrates how nostril curves are determined, which can be used to customize nasal fittings on a mask model. FIG. 11A shows a surface model where the nostril curves are determined and marked. FIG. 11B is a shaded model of a user's face where the nostril shapes are highly contrasted with remainder of the user's face. These high contrast properties can also appear in photographic images.

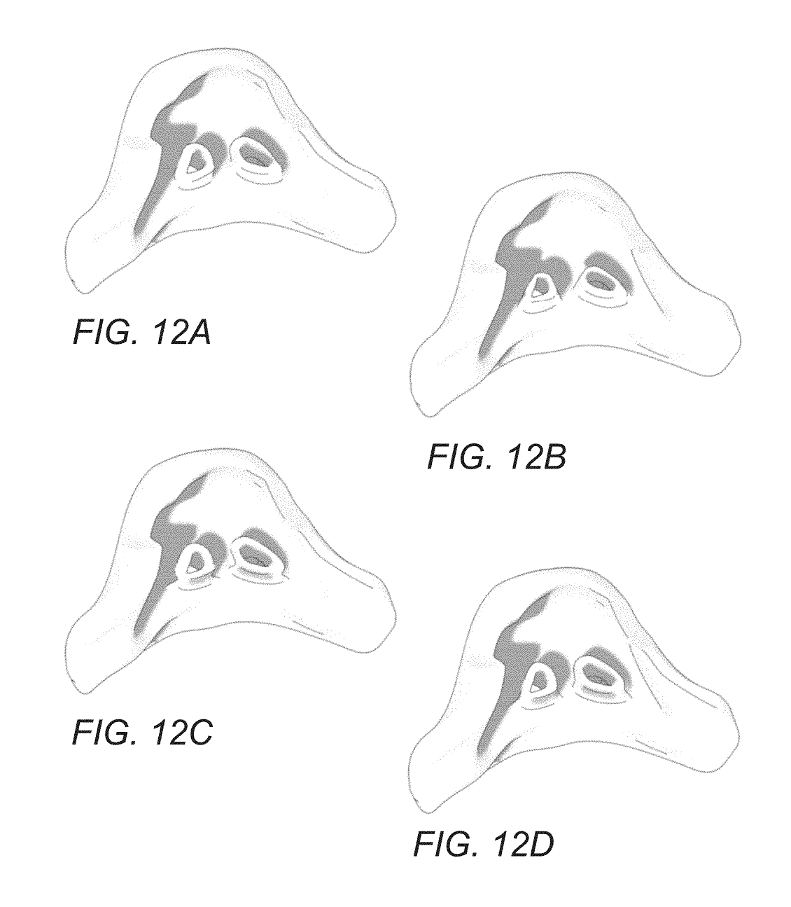

Nasal fittings can be designed to incorporate any shape, including but not limited to irregular shapes and asymmetric shapes. The nasal fittings can also be asymmetrical in the sense that the left and right nasal fittings can differ from one another for a single user. FIG. 12A-12D show various nasal fittings of different shapes and sizes. As shown, most nasal fittings are asymmetrical in both the individual nasal fitting's shape and between the left and right nasal fittings.

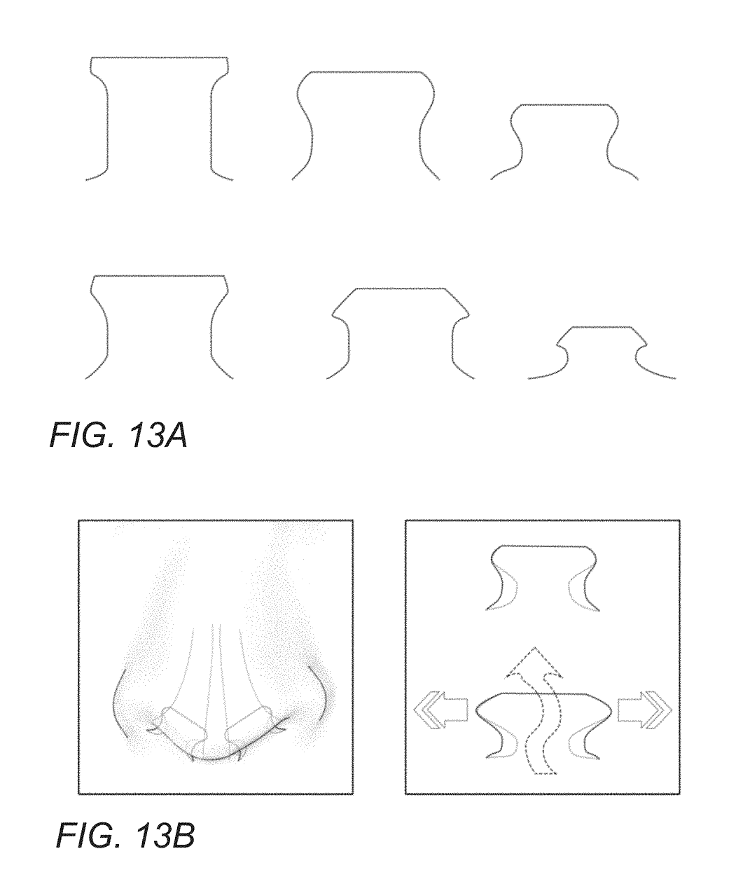

Nasal fittings in accordance with many embodiments of the invention can be designed to incorporate features that assist in providing a secure fit to the user's nostrils. Nasal fittings can be designed to include flanges that help prevent the nasal fittings from dislodging once seated inside the user's nostrils. FIG. 13A illustrates several different nasal fittings design in accordance with various embodiments of the invention. As shown, nasal fittings can be of different lengths and widths to accommodate different nostril types. Nasal fittings heights can be constraint to stay below a user's mucosal membrane. Furthermore, flanges can also be of different shapes and slopes. The different tapers can be adjusted to each individual user's nostrils to optimize fit. The shape and, in some cases, wall thickness, of the nasal fittings can be designed to expand at strategic locations when positive pressure is applied. In many embodiments, the flange is designed to further expand when positive air pressure is introduced. This expansion can push the flange against the nostril's interior wall to further help prevent dislodgement. This effect is conceptually illustrated in FIG. 13B.

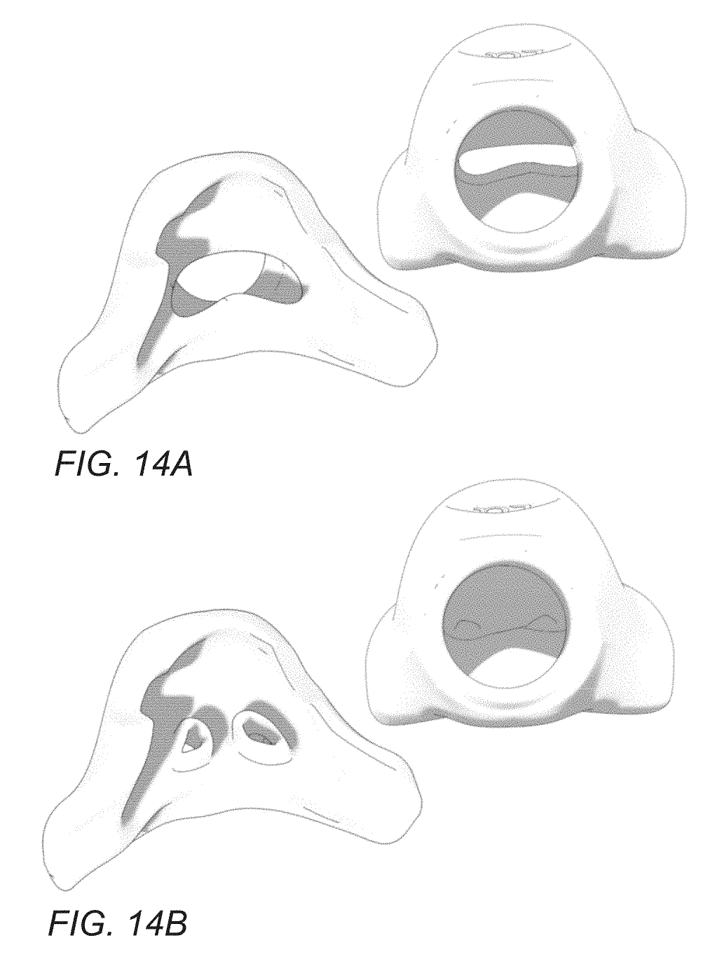

Nasal fittings can have different widths for a custom fit for a particular user. In many embodiments, the nasal interface can be designed as a single slit, shown in FIG. 14A. This design can be used in cases where a user's nostrils are too small. Due to minimum wall thickness fabrication constraints, fabricating a dual-pronged nasal interface, shown in FIG. 14B, customized to small nostrils can cause the resulting aperture size (inner diameter) to be too small for effective air flow.

Although nasal fittings are described above with respect to methods of modeling nasal curves, a person having ordinary skill in the art would appreciate that the discussions above also applies to customized masks with nasal fittings formed through the modeling methods above. As such, various embodiments of the invention are directed towards masks having nasal fittings with contours to match a specific user's nostril shapes.

### CPAP Mask Configuration

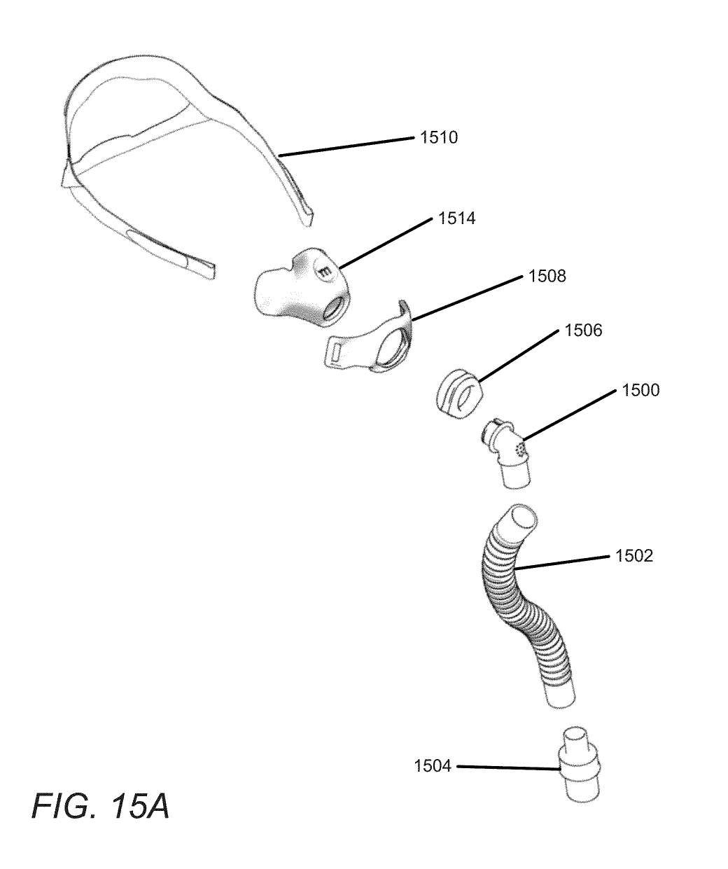

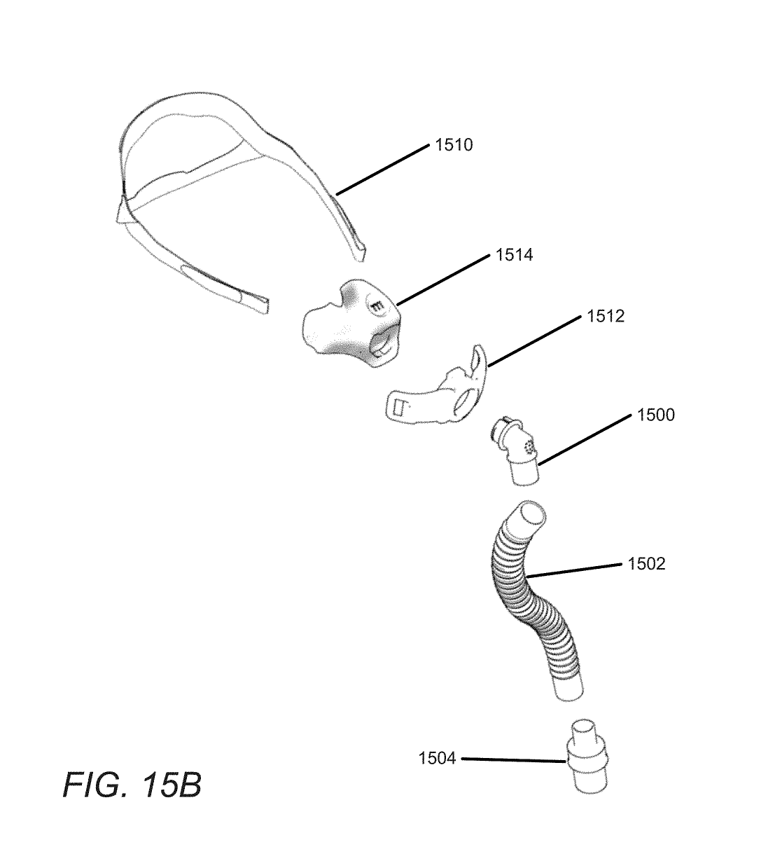

Customized CPAP masks in accordance with embodiments of the invention can be adapted into different configurations. The customized CPAP masks can be designed and manufactured to be readily incorporated into current CPAP ventilation systems. FIGS. 15A and 15B illustrates exploded views of two CPAP mask configurations in accordance with some embodiments of the invention. As shown, both systems include off the shelf components of current CPAP systems, such as the ventilated elbow 1500, the tube 1502, and the adapter 1504. FIG. 15A further shows a tube gasket 1506. In many embodiments, the tube gasket 1506 is designed to be asymmetrical in shape to help lock the tube gasket 1506 in place with the tube frame 1508. The tube frame 1508 can be designed and fabricated in a number of way. In some embodiments, the tube frame 1508 is designed to be customized to a user's face such that the strap 1510 can be attached in an ideal manner. The tube frame 1508 can be manufactured using any of a number of methods, such as but not limited to additive manufacturing process and injection molding. In the configuration shown in FIG. 15B, the tube gasket and tube frame functions as one piece 1512. Different configurations in accordance with various embodiments of the invention can include customized CPAP masks 1514. As described above, the customized CPAP masks 1514 can be modeled and manufactured with any of the custom features described above.

Although specific CPAP masks and related mask modeling algorithms, many different mask configurations and modeling algorithms can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for generating a surface model of a CPAP mask, the method comprising:
obtaining facial scan data of a user's face;
determining a set of landmark points from the facial scan data, wherein the set of landmark points relate to anatomical locations on the user's face;
generating a base model; and
deforming the base model into a deformed model using the set of landmark points.

2. The method of claim 1, further comprising generating a customized model by altering the deformed model using the facial scan data.

3. The method of claim 2, wherein altering the deformed model comprises performing a Boolean operation using the deformed model and a surface model generated using the facial scan data to alter an inner boundary surface of the deformed model such that the inner boundary surface of the deformed model can sit flush against the user's face.

4. The method of claim 2, further comprising manufacturing the customized model using an additive manufacturing process.

5. The method of claim 2, further comprising generating a mold model based on the customized model.

6. The method of claim 1, wherein the base model is a rigged model comprising a plurality of joints that deforms the base model when moved.

7. The method of claim 6, wherein deforming the base model comprises moving at least one of the plurality of joints based on a predefined relationship with at least one of the set of landmark points.

8. The method of claim 7, wherein the base model is deformed in adherence to a set of constraints.

9. The method of claim 1, wherein the base model comprises an averaged model obtained from an aggregate database, wherein the averaged model is an average of stored facial scan data in the aggregate database.

10. The method of claim 9, wherein the averaged model is an average of stored facial scan data of a selected demographic.

11. The method of claim 1, further comprising determining a nasal curve from the facial scan data and altering nasal fittings of the deformed model to match the nasal curve.

12. The method of claim 11, wherein determining a nasal curve comprises manually determining the nasal curve.

13. The method of claim 1, wherein the deformed model comprises a nasal interface comprising a single slit.

14. The method of claim 1, wherein the deformed model comprises a nasal interface comprising asymmetric nasal fittings.

15. The method of claim 14, wherein the asymmetric nasal fittings each comprises a flange.

16. The method of claim 1, wherein obtaining facial scan data comprises scanning the user's face using scanning technology.

17. The method of claim 1, wherein obtaining facial scan data comprises retrieving the facial scan data from a remote server.

18. The method of claim 1, wherein determining a set of landmark points comprises manually determining the set of landmark points.

19. The method of claim 1, wherein determining a set of landmark points comprises using an algorithm and machine vision to automatically determine the set of landmark points.

20. A customized CPAP mask comprising:
a customized mask comprising asymmetric nasal fittings and an inner boundary surface customized to sit flush with a face of a user;
a tube frame connected to the customized mask, wherein the tube frame comprises slots for strap attachment;
and a tube gasket connected to the tube frame, wherein the tube gasket is asymmetrical.

21. The customized CPAP mask of claim 20, wherein the asymmetric nasal fittings comprises a flange, wherein the flange is designed to expand when positive pressure is applied to the inside of the nasal fittings.

22. The customized CPAP mask of claim 20, wherein the customized mask further comprises a gasket side, wherein the gasket side houses the connection point for the tube frame, and wherein the center perpendicular line of the gasket side crosses a specific distance above a top ear point of the user when the customized mask is worn by the user.

23. The customized CPAP mask of claim 20, wherein the customized mask further comprises a bottom surface, wherein the bottom surface meets the face of the user at a point between the user's upper lip and columella when the customized mask is worn by the user.
